(19) United States
(12) Patent Application Publication
(10) Pub. No.: US 2022/0042037 A9
(43) Pub. Date: Feb. 10, 2022

(54) Adult Stem Cell Line Introduced with Hepatocyte Growth Factor Gene and Neurogenic Transcription Factor Gene with Basic Helix-Loop-Helix Motif and Uses Thereof

(71) Applicant: CELL&BRAIN CO., LTD., Jeonju-si (KR)

(72) Inventors: Hae Young Suh, Seongnam-si (KR); Sung Soo Kim, Seoul (KR); Seung Wan Yoo, Suwon-si (KR); Young Don Lee, Suwon-si (KR)

(21) Appl. No.: 17/216,169

(22) Filed: Mar. 29, 2021

Related U.S. Application Data
(63) Continuation-in-part of application No. 16/688,434, filed on Nov. 19, 2019.
Application No. 16/688,434 is a continuation-in-part of application No. 14/119,788, filed on Nov. 22, 2013.

(30) Foreign Application Priority Data
May 23, 2011 (KR) 10-2011-0048628

(51) Int. Cl. C12N 15/85; A61K 35/28; A61K 35/30; C12N 5/0775; C12N 15/86

(52) U.S. Cl. CPC C12N 15/85; A61K 35/28; A61K 35/30; C12N 2510/00; C12N 15/86; C12N 2501/60; C12N 2501/12; C12N 5/0663

(57) ABSTRACT

The present invention relates to an adult stem cell line introduced with an HGF gene and a neurogenic transcription factor gene of a bHLH family, a preparation method of the adult stem cell line, and a method for treating neurological diseases comprising the step of transplanting the adult stem cell line to a subject having neurological diseases. The adult stem cells according to the present invention, which are introduced with an HGF gene and a neurogenic transcription factor gene of a bHLH family, can be used to treat chronic impairment caused by cell death following stroke. Thus, the adult stem cells can be developed as a novel therapeutic agent or widely used in clinical trial and research for cell replacement therapy and gene therapy that are applicable to neurological diseases including Parkinson's disease, Alzheimer disease, and spinal cord injury as well as stroke.

### CROSS-REFERENCES TO RELATED APPLICATION

This application is a continuation-in-part of U.S. application Ser. No. 16/688,434, filed Nov. 19, 2019, which is a divisional of U.S. application Ser. No. 14/119,788, filed Nov. 22, 2013, which is a National Stage of international Application No. PCT/KR2012/004082, filed May 23, 2012, which designates the United States and which claims the benefit of and priority to Korean Patent Application NO 10-2011-0048628, filed May 23, 2011, the entirety of each of which is incorporated herein by specific reference. This application is also a continuation-in-part of U.S. application Ser. No. 14/119,788, filed Nov. 22, 2013, which is a National Stage of international Application No. PCT/KR2012/004082, filed May 23, 2012, which designates the United States and which claims the benefit of and priority to Korean Patent Application NO 10-2011-0048628, filed May 23, 2011, the entirety of each of which is incorporated herein by specific reference.

### BACKGROUND

### Technical Field

The present invention relates to an adult stem cell line, modified (or genetically modified) by introducing a gene encoding a hepatocyte growth factor (HGF) and a gene encoding a neurogenic transcription factor of a basic helix-loop-helix (bHLH) family into an adult stem cell line and uses thereof, and more particularly, to an adult stem cell line introduced with a hepatocyte growth factor gene and a neurogenic transcription factor gene of a basic helix-loop-helix family, a preparation method of the modified (or genetically modified) adult stem cell line, a composition for the prevention or treatment of neurological diseases comprising the modified (or genetically modified) adult stem cell line, and a method for treating neurological disease(s) comprising the step of administering the composition or the modified (or genetically modified) adult stem cell line to a subject having a neurological disease, and more specifically to treating stroke, AD (Alzheimer's), and/or ALS (muscular atrophic lateral sclerosis), or the effects or symptoms thereof.

### Related Technology

Mesenchymal stem cell (MSC) are stroma cells that help hematopoiesis in the bone marrow and have the ability to differentiate into a variety of mesodermal lineage cells, including osteocytes, chondrocytes, adipocytes, and myocytes, while also maintaining a pool of undifferentiated stem cells, and thus have gained prominence as a cell source for artificial tissues.

As MSCs have been reported to have a potential to differentiate into neuroglial cells in the brain, it has been proposed that MSCs can be utilized as sources for the treatment of neurological diseases in the central nervous system.

Several growth factors or hormones have been known to induce differentiation of undifferentiated cells into artificial neuronal cells. Unfortunately, those methods have a problem of generating non-neuronal cells together with neuronal cells, and the problem is more pronounced when the cells are transplanted into this brain of experimental animals. Thus, a need has existed to develop a direct method of inducing differentiation of MSCs into neuronal cells.

Neurogenin, also called NeuroD, is a transcription factor belonging to the basic helix-loop-helix (bHLH) family that plays important roles in the formation of the nervous system, and forms a complex with other bHLH proteins such as E12 or E47 to bind to DNA sequences containing the E-box (CANNTG) or on rare occasions, DNA sequences containing N-box. This binding has been found to be critical for bHLH proteins to activate tissue-specific gene expression that promotes neuronal differentiation.

The present inventors have endeavored to develop a stable material that effectively differentiates MSCs into neuronal cells. As a result, they have unexpectedly found that MSCs transduced with bHLH transcription factors such as neurogenin and neuroD can continuously express the bHLH transcription factors; and that the MSCs expressing the bHLH transcription factors can be transdifferentiated into a high level of neuronal cells when transplanted into the brain of experimental animals. On the basis of this finding, they reported that differentiation of MSCs into neuronal cells was induced to obtain excellent therapeutic effects in animal models of stroke, compared with non-induced MSCs (Korean Patent NO 10-0519227).

HGF, also known as scatter factor, is known to be a heparin-binding glycoprotein that has a strong anti-fibrotic activity in organs such as liver or kidney (Silver et al., Nat. Rev. Neurosci., 5:146-156, 2004). Studies of hepatocyte growth factor for the treatment of neurological diseases including stroke and spinal cord injury are now in progress. Its therapeutic effects on acute diseases have been reported, but a successful outcome on chronic diseases has not been reported yet.

### BRIEF SUMMARY

### Technical Problem

Without being bound to any particular theory, the use of MSCs in the treatment of neurological diseases can be advantageous in that it is possible to use autologous cells rather than heterologous cells. In a practical therapeutic procedure, however, the method has a disadvantage of requiring 2 to 4 weeks for isolation and cultivation of autologous cells and gene transfection, until autologous cell therapy after onset of stroke. Therefore, to address the problem of the time-consuming clinical procedure of autologous cell transplantation after the onset of stroke, studies have been made to develop a method of verifying and maximizing the therapeutic efficacies of autologous cells on chronic injuries.

The present inventors have made many efforts to develop a therapeutic composition and related method of treating neurological disease, and more specifically to treating stroke (e.g., chronic stroke), AD (Alzheimer's), and/or ALS (muscular atrophic lateral sclerosis), or the effects or symptoms thereof. As a result, they found that MSCs introduced with MSC/Ngn1+HGF showed therapeutic effects when transplanted into animal models of stroke, AD (Alzheimer's), and ALS (muscular atrophic lateral sclerosis) respectively. More generally, MSCs introduced with a bHLH transcription factor neurogenin 1 continuously express the bHLH transcription factor, and the MSCs further introduced with HGF showed therapeutic effects when transplanted into animal models of stroke, AD, and ALS, respectively.

### Solution to Problem

An object of the present invention is to provide a modified (or genetically modified) stem cell or stem cell line, preferably a modified (or genetically modified) adult stem cell or stem cell line, more preferably a modified (or genetically modified) adult, bone marrow derived stem cell or stem cell line, still more preferably a modified (or genetically modified) adult, mesenchymal stem cell or stem cell line, having introduced therein, or modified by introducing therein, a gene encoding a hepatocyte growth factor (HGF) and a gene encoding a neurogenic transcription factor of a basic helix-loop-helix (bHLH) family.

Another object of the present invention is to provide a/the modified (adult, etc.) stem cell line, or a stem cell (line) comprising a gene encoding a hepatocyte growth factor (HGF) and a gene encoding a neurogenic transcription factor of a basic helix-loop-helix (bHLH) family, or introduced therein.

Another object of the present invention is to provide a preparation method of the modified adult stem cell line.

Still another object of the present invention is to provide a method of administering the composition or modified adult stem cell line to a subject.

Still another object of the present invention is to provide a method for treating (e.g., reversing, or attenuating or preventing the progression of) neurological diseases, and more specifically, stroke, AD, and/or ALS, respectively, comprising administering (e.g., transplanting) the modified adult stem cell line to a subject having neurological diseases.

### Advantageous Effects

The adult stem cells according to the present invention, which are introduced with an HGF gene and a neurogenic transcription factor gene of a bHLH family, can be used to overcome chronic impairment caused by cell death following stroke. Thus, the adult stem cells can be developed as a novel therapeutic agent or widely used in clinical trial and research for cell replacement therapy and gene therapy that are applicable to neurological diseases including Parkinson's disease, Alzheimer disease, and spinal cord injury as well as stroke.

### BRIEF DESCRIPTION OF DRAWINGS

FIGS. 1A-1D is photographs showing the differentiation of MSCs into adipocytes, chondrocytes, and osteocytes, in which FIG. 1A is a photograph of adipocytes differentiated from MSCs, stained with oil red O, FIG. 1B is a photograph of chondrocytes differentiated from MSCs, stained with alcian blue, and FIGS. 1C and 1D are photographs of osteocytes differentiated from MSCs, stained with alkaline phosphatase and von Kossa, respectively.

FIG. 2A is a schematic representation a retroviral vector containing human neurogenin 1 gene and FIG. 2B is the result of Western blotting (lower panel) showing the expression of human neurogenin 1 in 293T cells that were introduced with a retroviral vector (upper panel) containing human neurogenin 1 gene.

FIG. 3 is the result of immunohistochemical staining using anti-neuronal marker TuJ1 (Beta-Tubulin-III) antibody to examine neurogenic differentiation of MSCs at two weeks after the human neurogenin 1 gene-introduced MSCs (hereinafter referred to as MSC/Ngn1) were infected with GFP-expressing adenovirus and transplanted into the striatum of albino rat.

FIG. 4 is the result of Western blot analysis showing the expression of intracellular (cell lysate) and extracellular (conditioned-medium; CM) HGF in MSCs introduced with adenoviral vector expressing human HGF (hereinafter referred to as MSC/HGF).

FIG. 5 is a photograph showing the result of immunocytochemistry to examine the expression level of HGF in MSC/HGF that were introduced with serially diluted adenoviral vector expressing human HGF.

FIGS. 6A-6C are photographs showing the expression of Ngn1 and HGF. FIG. 6A shows the Ngn1 expression by RT-PCR analysis. FIG. 6B is the result of Western blot analysis showing the expression of HGF in the cells transduced with Adenoviral vector encoding HGF. FIG. 6C is the immunocytochemistry to examine the expression of HGF.

FIG. 7A is a schematic presentation of transplantation. Ischemic stroke was induced by MCAo (occlusion of middle cerebral artery) and the cells were transplanted at indicated time. Eight weeks later (8w), neurological scores were assessed. FIG. 7B is a graph summarizing the therapeutic efficacy of the human HGF gene and human neurogenin 1 gene-introduced MSCs (hereinafter referred to as MSC/Ngn1+HGF) in stroke animal model according to the cell transplantation time. (*p<0.05, *8*p<0.01 compared to the PBS control)

FIG. 8A is a schematic presentation of experiments. FIG. 8B and FIG. 8C are graphs showing the results of animal behavioral tests including Adhesive Removal Test (FIG. 8B left panel) and Rotarod Test (FIG. 8C right panel) to evaluate the therapeutic efficacy of MSC/Ngn1+HGF in stroke animal model.

FIG. 9A is photographs showing the results of a MRI (upper panel) and FIG. 9B illustrates quantitative analysis of the infarct volume (lower panel) to evaluate the therapeutic efficacy of MSC/Ngn1+HGF in stroke animal model.

FIG. 10 is a photograph showing the result of immunohistochemistry using antibodies specific for GFAP and MAP2 to examine glial scar (GFAP) and survival of neuronal cells (MAP2) in the peri-infarct region 3 months after MCAo.

FIG. 11 is a photograph showing the brain inflammation (Iba1+microglia) in the ischemic brain. FIG. 11B is a graph showing the IBA1-positive immunoreactivity, which was reduced following any types of transplantation (MSC, MSC/Ngn1, MSC/HGF, and MSC/Ngn1+HGF) compared to the PBS control. (*: p<0.05 compared to the PBS control). FIG. 11C is a schematic presentation of the antiinflammation.

FIG. 12 is a photographs showing astrocytic glial scar (GFAP+ reactive astrocyte) in peri-infarct region of the animals that were sacrificed at 3 months after MCAo. FIG. 12B is a representative photograph showing the peri-infarct region. FIG. 12C illustrates the relative intensity of GFAP (red) from 3 animals per group. FIG. 12D is a schematic presentation of the anti-gliosis effects of MSC/Ngn1+HGF.

FIG. 13A is a photograph showing distribution of blood vessels in the brain of the animals that were sacrificed at 3 months after MCAo. FIG. 13B is a photograph showing the area of interest in the peri-infarct region of the striatum and cortex. FIG. 13C illustrates relative intensity of Tomato lectin labeled-blood vessels in the striatum and cortex. FIG. 13D is a schematic presentation of the pro-angiogenic effect of MSC/Ngn1+HGF.

FIG. 14A is a photograph showing proliferation of endogenous neuoblasts in a chronic stroke model. Proliferating Dcx-positive neuroblasts uptake BrDU (a thymidine analogue). FIG. 14B illustrates that the number of Dcx+ (Doublecortin-positive) neuroblasts were significantly increased in the striatum of the animals transplanted with MSC/Ngn1+HGF, that the effects of MSC and MSC/Ngn1 were minimal, while MSC/HGF were less effective to increase DCx+ cells in a chronic stroke model. FIG. 14C is a schematic presentation of the pro-neurogenic effects of MSC/Ngn1+HGF.

FIG. 15A is a photograph showing the cells expressing MSC/Ngn1+HGF remain 0028, and occasionally trans-differentiated into neurons. FIG. 15B are photographs illustrating that MSC/Ngn1+HGF (green) were occasionally positive for Synasin 1 (a synaptic marker). FIG. 15C is a schematic presentation of trans-differentiation of MSC/Ngn1+HGF.

FIG. 16 summarizes the mode of actions of MSC/Ngn1+HGF in the chronic stroke model. “1˜4” are the effects of MSC/Ngn1+HGF on endogenous mouse cells in the stroke brain. The effect of “5” is trans-differentiation of transplanted MSC/Ngn1+HGF into neuronal cells. MSC/Ngn1+HGF improves functional recovery as shown in FIGS. 7-9.

FIG. 17A is a graph showing that transplantation of MSC/Ngn1+HGF cells into tail vein is effective to delay the disease progression and thereby increase the survival of the amyotrophic lateral sclerosis (ALS) model. FIG. 17B is a summary showing that both the means and median was increased by the transplantation of the cells.

FIG. 18A is a photograph showing the ventral motor neurons are preserved by MSC/Ngn1+HGF in the spinal cord in ALS mouse model. FIG. 18B is a summary graph showing the number of healthy ventral motor neurons.

FIG. 19A is a graph showing that the results of Morris water maze test performed 6 weeks after cell transplantation of MSC/Ngn1+HGF in 5XFAD, an Alzheimer mouse model. FIG. 19B is a graph showing that the swim speeds were not significantly different in 4 groups.

FIG. 20A is a photograph showing that the (3-amyloid plaque deposition (thioflavin+) in the mouse brain sacrificed after Morris water maze test in 5xFAD mice. FIG. 20B is a bar graph showing the thioflavin-positive pixels obtained from 12 brain sections from three animals per group.

FIG. 21A is a photograph that shows TUNEL-positive, apoptotic cell death in cortex, hippocampus, striatum, and thalamus of 5xFAD mice shown in FIG. 21B after transplantation. FIG. 21C is a summary graph showing that apoptotic cell death is most effectively prevented by MSC/Ngn1+HGF. FIG. 21B is a photograph of a parasagittal section of the brain.

### BEST MODE FOR CARRYING OUT THE INVENTION

In one aspect of the present invention, the present invention provides an adult stem cell line, modified (or genetically modified) by introducing a gene encoding a hepatocyte growth factor (HGF) and a gene encoding a neurogenic transcription factor of a basic helix-loop-helix (bHLH) family into an adult stem cell line.

As used herein, the term “adult stem cell” means an undifferentiated cell that can differentiate into specialized cell types of the tissue if needed. The adult stem cell line is, but is not particularly limited to, preferably, a stem cell or stem cell line derived from bone marrow, adipose tissue, blood, umbilical cord blood, umbilical cord, adipose tissue, liver, skin, gastrointestinal tract, muscle, placenta, uterus or aborted fetuses, more preferably a bone marrow-derived adult stem cell line, and most preferably a bone marrow-derived mesenchymal stem cell (MSC) or MSC line. Bone marrow-derived adult stem cell can include a variety of adult stem cells such as MSCs and hematopoietic stem cells capable of producing blood cells and lymphocytes. Among them, MSCs are able to easily proliferate ex vivo and differentiate into a variety of cell types (adipocytes, chondrocytes, myocytes, and osteocytes). Thus, they can be used as a useful target in gene and cell therapy, but the use thereof is not particularly limited. Both autologous and allogeneic adult stem cells can be used. In a preferred embodiment of the present invention, bone marrow of a healthy person donated in the bone marrow bank was used.

As used herein, the term “Hepatocyte Growth Factor (HGF)”, also known as scatter factor, means a multifunctional heterodimeric polypeptide produced by mesenchymal cells. The HGF is composed of a 69 kDa alpha-chain containing the N-terminal finger domain and four Kringle domains, and a 34 kDa beta-chain which has a similarity to protease domains of chymotrypsin-like serine protease. Human HGF is synthesized as a biologically inactive single chain precursor consisting of 728 amino acids. Biologically active HGF is achieved through cleavage at the R494 residue by a specific serum serine protease. The active HGF is a heterodimer which is composed of 69 kDa alpha-chain and 34 kDa beta-chain linked via a disulfide bond. In the present invention, the HGF is introduced into the adult stem cell line to obtain a transduced cell line. A nucleotide sequence encoding the preferred HGF is known (GenBank Accession NO NM_000601.4 166-2352, or BC130286.1 (76-2262)).

As used herein, the term “Basic Helix-Loop-Helix (bHLH)” expresses the shape of transcription factors, and refers to a form of two helices connected by a loop. The bHLH transcription factors are known to play important roles in gene expression of multi-cellular organisms.

The bHLH transcription factors are, but are not particularly limited to, preferably neurogenic transcription factors, and more preferably neurogenin 1 gene (GenBank Accession No: U63842, U67776), neurogenin 2 gene (GenBank Accession No: U76207, AF303001), neuro D1 gene (GenBank Accession No: U24679, AB018693), MASH1 gene (GenBank Accession No: M95603, L08424), MATHS gene (GenBank Accession No: D85845), E47 gene (GenBank Accession No: M65214, AF352579) or the like. Moreover, the neurogenic transcription factor having an alteration, a deletion, or a substitution in a part of the polynucleotide sequence may be used, as long as it shows an activity equivalent or similar to that of the neurogenic transcription factor. In a preferred embodiment of the present invention, an adult stem cell line into which a hepatocyte growth factor gene and a neurogenin 1 gene were introduced was prepared.

The MSCs introduced with the bHLH transcription factor gene have the potential to differentiate into neuronal cells rather than the potential to differentiate into osteocytes, myocytes, adipocytes, and chondrocytes, and they are able to differentiate into neuronal cells under particular conditions in vitro. According to one Example of the present invention, MSC/Ngn1+HGF were prepared, and they were found to effectively differentiate into neuronal cells when transplanted into the brain tissue of experimental animals.

As used herein, the term “modified” may be synonymous with “genetically modified” unless context clearly dictates otherwise.

As used herein, the term “adult stem cell line introduced with the HGF gene and the neurogenic transcription factor gene of the bHLH family” refers to an adult stem cell line that is introduced with the above described HGF gene and neurogenic transcription factor gene of the bHLH family, preferably an adult stem cell line that is introduced with the HGF gene of SEQ ID NO 1 and the neurogenin 1 gene of SEQ ID NO 2. However, the adult stem cell line is not particularly limited thereto, as long as it retains the ability to differentiate into neuronal cells.

With respect to the objects of the present invention, it is preferable that the HGF gene is cloned into a vector, and then introduced into the adult stem cell.

As used herein, the term “vector”, which describes an expression vector capable of expressing a target protein in a suitable host cell, refers to a genetic construct that includes essential regulatory elements to which a gene insert is operably linked in such a manner as to be expressed.

As used herein, the term “operably linked” refers to a functional linkage between a nucleic acid sequence coding for the desired protein and a nucleic acid expression control sequence in such a manner as to allow general functions. The operable linkage may be prepared using a genetic recombinant technique that is well known in the art, and site-specific DNA cleavage and ligation may be carried out using enzymes that are generally known in the art.

The vector is, but is not particularly limited to, preferably a plasmid vector, a cosmid vector, a viral vector, and more preferably, viral vectors derived from HIV (Human immunodeficiency virus), MLV (Murine leukemia virus), ASLV (Avian sarcoma/leukosis), SNV (Spleen necrosis virus), RSV (Rous sarcoma virus), MMTV (Mouse mammary tumor virus), MSV (Murine sarcoma virus), adenovirus, adeno-associated virus, herpes simplex virus or the like.

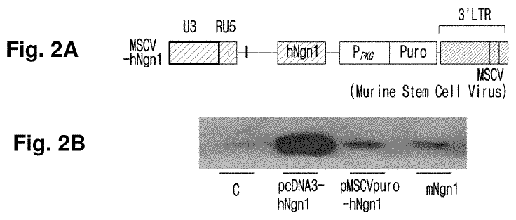

According to one Example of the present invention, for the introduction of neurogenin 1 gene, the coding region (55-768 bp) in the gene sequence of GenBank Accession NO U63842 of FIG. 2 was cloned into a pMSCV-puro plasmid to prepare a recombinant vector pMSCV/puro-hNgn1, and the obtained recombinant vector was introduced into a cell line producing retrovirus to prepare a retroviral vector. Then, the obtained retroviral vector was introduced into a bone marrow-derived MSC line to prepare a transduced adult stem cell.

According to another Example of the present invention, for the introduction of HGF gene, the coding region (166-2352 bp) in the gene sequence of GenBank Accession NO NM 000601.4 was cloned into pShuttle-CMV, and then a recombinant vector pAd-HGF was prepared by recombination with pAdEasy-1. The recombinant vector was linearized by cleavage with the restriction enzyme PacI, and the linearized recombinant vector was introduced into a cell line producing adenovirus to prepare an Adeno-HGF vector. Then, the obtained Adeno-HGF vector was introduced into a bone marrow-derived MSC line to prepare a transduced adult stem cell.

The gene introduction into the adult stem cell of the present invention is, but is not particularly limited to, performed by transduction, and the transduction may be readily performed by the typical method known in the art.

As used herein, the term “transformation” refers to artificial genetic alteration by introduction of a foreign DNA or a foreign DNA-containing viral vector into a host cell, either as an extrachromosomal element, or by chromosomal integration. Generally, the transformation method includes infection using retrovirus and adenovirus, CaCl₂ precipitation of DNA, a Hanahan method that is an improved CaCl₂ method by using dimethylsulfoxide (DMSO) as a reducing material, electroporation, calcium phosphate precipitation, protoplastfusion, agitation using silicon carbide fiber, Agrobacterium-mediated transduction, PEG-, dextransulfate-, lipofectamine-, and desiccation/inhibition-mediated transduction. According to one example of the present invention, transduction was performed by introduction of the retroviral vector containing neurogenin and the Adeno-HGF vector containing HGF gene into stem cells.

In the case of a vector containing the polynucleotide, it is preferable to contain 10³ to 10¹² IU (10 to 10¹⁰ PFU/ml), more preferably to contain 10⁵ to 10¹⁰ IU. Most preferably, the adenovirus transfection can be carried out by adding the adenovirus solution having a titer of 10³ to 10⁸ PFU/ml.

In another aspect, the present invention provides a preparation method of the modified, adult stem cell line, or adult stem cell line that is introduced with the HGF gene and the neurogenin 1 gene.

As described above, the type of the adult stem cell line introduced with the HGF gene and the neurogenin 1 gene is not particularly limited, and any cell line may be used as the cell line of the present invention, as long as it has the potential to differentiate into the specialized cell types of the tissue.

Preferably, the adult stem cell line may be an adult stem cell line derived from bone marrow, adipose tissue, blood, umbilical cord blood, umbilical cord, adipose tissue, liver, skin, gastrointestinal tract, muscle, placenta, uterus or aborted fetuses. More preferably, the adult stem cell line is a bone marrow-derived adult stem cell line. Much more preferably, the adult stem cell line is a bone marrow-derived MSC line.

Introduction of a particular gene into a stem cell line (e.g., adult, mesenchymal, and/or bone marrow derived stem cell line) may be performed by using a transduction method. As described above, a typical transduction method known in the art may be used without limitation. According to one Example of the present invention, a transduced adult stem cell line was prepared by introduction of the MSCV-puro/hNgn1 and Adeno-HGF into the adult stem cell line. After transduction of MSCs with a retroviral vector (MSCV-puro/hNgn1 gene), puromycin was used for selection. After transfection of MSCs with Adeno-HGF, an HGF antibody was used to examine its expression, and multiplicity of infection (MOI) was determined and used.

The method of producing the adult, mesenchymal, and/or bone marrow-derived stem cell line introduced with HGF gene and neurogenin 1 gene of the present invention may include the following steps:

(a) introducing a gene coding hepatocyte growth factor having a nucleotide sequence of SEQ ID NO 1 and a gene coding neurogenin 1 having a nucleotide sequence of SEQ ID NO 2 into cultured adult stem cells;

(b) selecting the modified adult stem cell line that is introduced with both genes coding hepatocyte growth factor and neurogenin 1; and

(c) culturing the selected the modified adult stem cell line.

In the method of producing the modified, bone marrow-derived adult stem cell line that is introduced with HGF gene and neurogenin 1 gene, introducing the gene coding hepatocyte growth factor and the gene coding neurogenin 1 are performed sequentially or in reverse order, or simultaneously, but the order and method are not particularly limited.

According to one Example of the present invention, among the adult stem cells, bone marrow-derived MSCs were isolated. The isolated MSCs were cultured in a DMEM medium containing 10% FBS, 10 ng/mL bFGF, and 1% penicillin/streptomycin, and subcultured up to four passages for use in experiments.

In the step of transducing with the neurogenin 1 gene, the neurogenin 1 gene was ligated to the pMSCV-puro vector using T4 DNA ligase, and transduced into E. coli DH5α. Finally, a pMSCV-puro/hNgn1 vector was prepared by insertion of hNgn1 gene into the pMSCV-puro vector. The pMSCV-puro/hNgn1 vector was introduced into 293T cells with gag/pol- and env-expression vectors or a retroviral packaging cell lines such as PA317 (ATCC CRL-9078) or PG13 (ATCC CRL-10686) according to the calcium phosphate precipitation method.

The resulting retroviral vector containing the neurogenin 1 gene was introduced into the subcultured cell line. The cells introduced with neurogenin 1 gene were subcultured in the medium containing 2 μg/mL of puromycin for 2 weeks so as to select the surviving cells introduced with neurogenin 1. Finally, a cell line continuously expressing neurogenin 1 was prepared by the above procedure.

In the step of transducing with the HGF gene, the HGF-cloned pShuttle-CMV-HGF and pAdEasy-1 were co-transduced into E.coli (BJ 5183 strain) by electroporation, and then cultured in a medium containing kanamycin (50 μg/mL) until colonies were formed. Plasmids were obtained from each colony, and candidate colonies were selected by standard restriction enzyme digestion. Base sequence was analyzed to obtain pAd-HGF. The pAd-HGF was linearized by cleavage with the restriction enzyme PacI, and introduced into HEK293 cell by calcium phosphate precipitation to obtain a culture broth containing Adeno-HGF virus. In order to select a MSC line where HGF was successfully introduced, protein expression of HGF was examined by immunocytochemical staining and western blotting analysis using an antibody against HGF (FIGS. 4˜6).

In still another aspect, the present invention provides the modified adult stem cell line, or adult stem cell line introduced with HGF gene and neurogenin 1 gene, for the prevention or treatment (e.g., reversing, or attenuating or preventing progression) of neurological diseases.

As used herein, the term “neurological diseases” refers to a variety of diseases associated with nerves, in particular, cranial nerves. The neurological diseases may be, but are not particularly limited to, Parkinson's disease, Alzheimer disease, Huntington's chorea, amyotrophic lateral sclerosis, epilepsy, schizophrenia, acute stroke, chronic stroke, or spinal cord injury, and preferably chronic stroke.

As used herein, the term “prevention” refers to all of the actions in which the occurrence of neurological diseases or diseases associated therewith is restrained or retarded by using the adult stem cell line introduced with HGF gene and neurogenin 1 gene.

As used herein, the term “treatment” refers to all of the actions in which the symptoms of neurological diseases or diseases associated therewith have taken a turn for the better or been modified favorably by using the adult stem cell line introduced with HGF gene and neurogenin 1 gene.

The MSCs introduced with HGF gene and neurogenin 1 gene of the present invention may exist in a form of a pharmaceutical composition including the MSCs for treatment.

Meanwhile, the composition of the present invention may be a pharmaceutical composition further including a pharmaceutically acceptable carrier. The composition including a pharmaceutically acceptable carrier may be prepared into parenteral formulation. Formulations may be prepared using diluents or excipients ordinarily employed, such as a filler, an extender, a binder, a wetting agent, a disintegrating agent, and a surfactant. Examples of the solid preparation include a tablet, a pill, a powder, a granule, and a capsule, and the solid preparation may be prepared by mixing one or more compounds with at least one excipient such as starch, calcium carbonate, sucrose, lactose, and gelatin. Further, in addition to the excipients, lubricants such as magnesium stearate and talc may be used. Examples of a liquid preparation include a suspension, a liquid for internal use, an emulsion, and a syrup, and various excipients such as a wetting agent, a sweetener, a flavor, and a preservative may be contained, in addition to general diluents such as water and liquid paraffin. Examples of the preparation for parenteral administration may include an aseptic aqueous solution, a non-aqueous solvent, a suspension, an emulsion, a lyophilized agent, and suppository. As the non-aqueous solvent and suspension, propylene glycol, polyethylene glycol, plant oil such as olive oil, and injectable ester such as ethyloleate may be used. As a suppository base, witepsol, macrogol, tween 61, cacao butter, lauric butter, glycerogelatin or the like may be used. The pharmaceutical composition may be formulated into any preparation selected from the group consisting of a tablet, a pill, a powder, a granule, and a capsule, a suspension, a liquid for internal use, an emulsion, and a syrup, an aseptic aqueous solution, a non-aqueous solvent, a suspension, an emulsion, a lyophilized agent, and suppository.

In still another aspect, the present invention provides a method for treating neurological diseases, comprising the step of administering (e.g., transplanting) the inventive composition, or modified, adult mesenchymal stem cells (MSCs) of the present disclosure, to a subject having neurological diseases or suspected of having neurological diseases (illustratively, directly into the brain of a subject having the neurological disease).

As used herein, the term “subject” refers to living organisms that have the nervous system and thus are susceptible to the above described neurological diseases caused by various factors, and preferably mammals.

As used herein, the term “mammal” refers to mouse, rat, rabbit, dog, cat, and especially human, and refers to any organism of the Class “Mammalia” of higher vertebrates that nourish their young with milk secreted by mammary glands.

In various embodiments, the composition of the present disclosure may be administered to a subject via any of the common routes, as long as it is able to reach a desired tissue. A variety of administration modes are contemplated, including intraperitoneally, intravenously, intramuscularly, subcutaneously, intradermally, intranasally, intrapulmonarily and intrarectally, but the present invention is not limited to these exemplified administration modes. In addition, the composition of the present invention may be used singly or in combination with hormone therapy, drug therapy and biological response regulators in order to exhibit antioxidant effects.

Moreover, the composition of the present invention may be administered in a pharmaceutically effective amount. As used herein, the term “pharmaceutically effective amount” refers to an amount sufficient for the treatment of diseases, which is commensurate with a reasonable benefit/risk ratio applicable for medical treatment. An effective dosage of the present composition may be determined depending on the subject and severity of the diseases, age, gender, drug activity, drug sensitivity, administration time, administration route, excretion rate, duration of treatment, simultaneously used drugs, and other factors known in medicine. The composition of the present invention may be administered as a sole therapeutic agent or in combination with other therapeutic agents, and may be administered sequentially or simultaneously with conventional therapeutic agents. This administration may be provided in single or multiple doses. Taking all factors into consideration, it is important to conduct administration of minimal doses capable of giving the greatest effects with no adverse effects, such doses being readily determined by those skilled in the art.

In addition, the composition of the present invention may be used singly or in combination with surgical operation, hormone therapy, drug therapy and biological response regulators in order to prevent or treat inflammatory diseases.

Hereinafter, the present invention will be described in more detail with reference to Examples. However, these Examples are for illustrative purposes only, and the invention is not intended to be limited by these Examples.

### Example 1

### Isolation and Culture of MSCs

### Example 1-1

### Isolation of MSCs

4 mL of HISTOPAQUE 1077 (Sigma) and 4 mL of bone marrow obtained from Bone marrow bank (Korean Marrow Donor Program, KMDP) were added to a sterilized 15 mL test-tube, and centrifugation was performed using a centrifuge at room temperature and 400×g for 30 minutes. After centrifugation, 0.5 mL of the buffy coat located in the interphase was carefully collected using a pasteur pipette, and transferred into a test-tube containing 10 mL of sterilized phosphate buffered saline (PBS). The transferred buffy coat was centrifuged at 250×g for 10 minutes to remove the supernatant and 10 mL of phosphate buffer was added thereto to obtain a suspension, which was centrifuged at 250×g for 10 minutes.

The above procedure was repeated twice and a DMEM medium (Gibco) containing 10% FBS (Gibco) was added to the resulting precipitate. A portion of the resulting solution corresponding to 1×10⁷ cells was placed in a 100 mm dish and incubated at 37° C. for 4 hours while supplying 5% CO2 and 95% air. The supernatant was then removed to eliminate cells that were not attached to the bottom of the culture dish, and a new medium was added to continue culturing.

### Example 1-2

### Culture of MSCs

The MSCs isolated in Example 1-1 were incubated in a CO₂ incubator kept at 37° C., while changing an MSC medium (10% FBS+10 ng/mL of bFGF (Sigma)+1% penicillin/streptomycin (Gibco)+89% DMEM) at an interval of 2 days. When the cells reached approximately 80% confluence, the cells were collected using 0.25% trypsin/0.1 mM EDTA (GIBCO) and diluted 20-fold with the medium, and then subcultured in the new dishes. The rest of cells thus obtained were kept frozen in medium containing 10% DMSO, and their potentials to differentiate into adipocytes, chondrocytes, and osteocytes were examined as follows.

### Example 1-3

### Adipogenic Differentiation

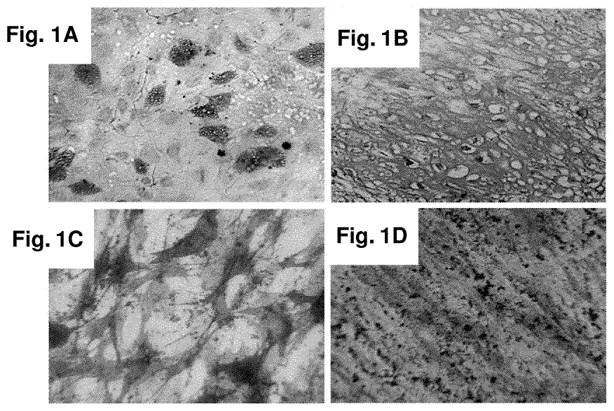

MSCs were cultured in the MSC medium for a predetermined period of time, followed by culturing in an adipogenic differentiation induction medium (DMEM medium containing 1 μM dexamethasone (Sigma), 0.5 μM methyl-isobutylxanthine (Sigma), 10 μg/mL of insulin (GIBCO), 100 nM indomethacin (Sigma) and 10% FBS) for 48 hours. The resulting mixture was subsequently incubated in an adipogenic maintenance medium (DMEM medium containing 10 μg/mL of insulin and 10% FBS) for 1 week and stained with oil red O (FIG. 1A). FIG. 1A is a photograph of adipocytes differentiated from MSCs, which were stained with oil red O. As shown in FIG. 1A, lipid droplets stained with red were observed inside the cells, indicating that MSCs were successfully differentiated into adipocytes.

### Example 1-4

### Chondrogenic Differentiation

MSCs were cultured in the MSC medium for a predetermined period of time, and 2×10⁵ of the cells were collected using trypsin and transferred into a test-tube, centrifuged, and then, re-incubated in 0.5 mL of a serum-free chondrogenic differentiation induction medium (50 mL of high-glucose DMEM (GIBCO), 0.5 mL of 100×ITS (0.5 mg/mL of bovine insulin, 0.5 mg/mL of human transferrin, 0.5 mg/mL of sodium selenate (Sigma), 50 μL linolenic acid-albumin (Sigma), 0.2 mM 100 nM dexamethasone, and 10 ng/mL of TGF-betal (Sigma)) for 3 weeks while replacing the medium every 3 days. Then, the cells were fixed with 4% paraformaldehyde, sectioned using a microtome, and then stained with alcian blue (FIG. 1B). FIG. 1B is a photograph of chondrocytes differentiated from MSCs, which were stained with alcian blue. As shown in FIG. 1B, the extracellular cartilage matrix was stained blue and the presence of chondrocytes in cartilage lacunae was observed, indicating that the MSCs were differentiated into chondrocytes.

### Example 1-5

### Osteogenic Differentiation

MSCs were cultured in the MSC medium for a predetermined period of time, followed by culturing in an osteogenic differentiation induction medium (DMEM containing 10 mM beta-glycerol phosphate (Sigma), 0.2 mM ascorvate-2-phosphate (Sigma), 10 nM dexamethasone and 10% FBS) for 2 weeks while replacing the medium every 3 days. Then, the cells were fixed with paraformaldehyde, and stained with von Kossa and alkaline phosphatase (AP) (FIGS. 1C and 1D). FIGS. 1C and 1D are photographs of osteocytes differentiated from MSCs, which were stained with alkaline phosphatase and von Kossa, respectively. As shown in FIGS. 1C and 1D, the extracellular accumulation of calcium minerals in the form of hydroxyapatite and the increase of the intracellular alkaline phosphatase activity suggest that the MSCs were differentiated into osteocytes.

### Example 2

### Construction and Expression of Retrovirus of Human Neurogenic Transcription Factor, Neurogenin 1

### Example 2-1

### Construction of Retroviral Vector Expressing Human Ngn 1

The sequence of SEQ ID NO 2 corresponding to the coding region (55-768 bp) in the U63842 gene sequence was ligated into a pMSCV-puro vector (Clontech) using T4 DNA ligase (Roche), and then transduced into E. coli DH5α to finally construct a pMSCV-puro/hNgn1 vector where human neurogenin 1 (hNgn1) gene was inserted into the pMSCV-puro vector. The constructed pMSCV-puro/hNgn1 vector was introduced into 293T cells with by calcium phosphate precipitation, and the expression was examined by Western blotting (FIG. 2). FIG. 2 is the result of Western blotting (lower panel) showing the expression of hNgn1 in 293T cells that was introduced with a retroviral vector (upper panel) containing hNgn1 gene.

### Example 2-2

### Preparation of Retrovirus Containing Neurogenin 1

The pMSCV-puro/hNgn1 vector was introduced into a retroviral packaging cell line, PA317 (ATCC CRL-9078) or PG13 (ATCC CRL-10686) according to the calcium phosphate precipitation method. After 48 hours, the culture solution was collected and filtered with 0.45 μm membrane to obtain retrovirus solution. The retrovirus solution was kept at −70° C. until use.

### Example 3

### Construction of MSC/Ngn1 and In Vivo Neuronal Differentiation

### Example 3-1

### Introduction of Neurogenin 1 into MSCs

MSCs were cultured to 70% confluence in 100 mm culture dishes. Added thereto was 4 mL of the neurogenin 1 retrovirus solution obtained in Example 2-2 which was mixed with polybrene (Sigma) to a final concentration of 8 μg/mL, and incubated for 8 hours. The retrovirus solution was then removed, and the MSCs were cultured in 10 mL of MSC medium for 24 hours, followed by re-infection of the retrovirus. The above procedure was repeated 1-4 times. Then, MSCs were collected using trypsin and diluted 20 fold with the medium. The obtained cells were subcultured in a medium supplemented with 2 μg/mL of puromycin (Sigma) for 2 weeks so as to select the surviving cells infected with retrovirus. Finally, MSCs having a puromycin resistance were used as MSC/Ngn1.

### Example 3-2

### Labeling of Cells for Transplantation

In order to examine whether neurogenin 1 gene increases the transplantation rate and neuronal differentiation, MSC/Ngn1 were infected with GFP-expressing adenovirus.

The adenovirus transfection was carried out by adding the adenovirus solution having a titer of 1×10⁸ PFU/mL with 100 MOI already described earlier for 3 hours. After adenovirus transfection, MSC/Ngn1 were collected using 0.25% trypsin/0.1% EDTA and diluted with PBS to 333 10³ cells per 1 μL.

### Example 3-3

### Transplantation

Transplantation was carried out using adult Sprague-Dawley albino female rats (250 g) (Dae Han Bio Link Co., Ltd) as follows:

Firstly, albino rats were anesthetized with an intraperitoneal injection of 75 mg/kg ketamine and 5 mg/kg rumpun, the fur at the incision region was removed, and then the ears and mouth were fixed to a stereotaxic frame. The vertex was sterilized with 70% ethanol and an approximately 1 cm incision was made. Subsequently, 1 μL of PBS containing 3×10³ of MSC/Ngn1 was put in a 10 μL Hamilton syringe, which was placed in a Hamilton syringe rack. After drilling at the exposed dura at positions of Bregma AP, +1.0; ML 3.0; LV, +4.0, 1 μL of the cells was injected at a rate of 0.2 μL/min using a Hamilton syringe. Twenty minutes after injection, the syringe was slowly removed. The incision was sutured using a sterilized thread and needle, and disinfected using a disinfectant. 5 mg/kg of an immunosuppressant cyclosporin A (Sigma) was daily administered by intraperitoneal injection until the brain was extracted.

### Example 3-4

### Preparation of Tissue Slice

Two weeks after transplantation, the albino rats were anesthetized with an intraperitoneal injection of 75 mg/kg ketamine and 5 mg/kg rumpun. The chests were opened, and perfusion wash-out was performed using saline through the left ventricle. Perfusion fixation was performed using paraformaldehyde in 0.1 M phosphate buffer solution (pH 7.4). The brains were extracted, and post-fixed in the same fixation solution at 4° C. for 16 hours. The post-fixed brain was deposited in 30% sucrose for 24 hours and sectioned using a sliding microtome with a thickness of 35 μm. The sections thus obtained were mounted to silane-coated slides (MUTO PUREW CHEMICAS CO., LTD, Japan) and stored at 4° C. in PBS until use. The tissue sections mounted on slides were dipped in 1×PBS/0.1% Triton X-100 for 30 minutes.

### Example 3-5

### Immunohistochemistry

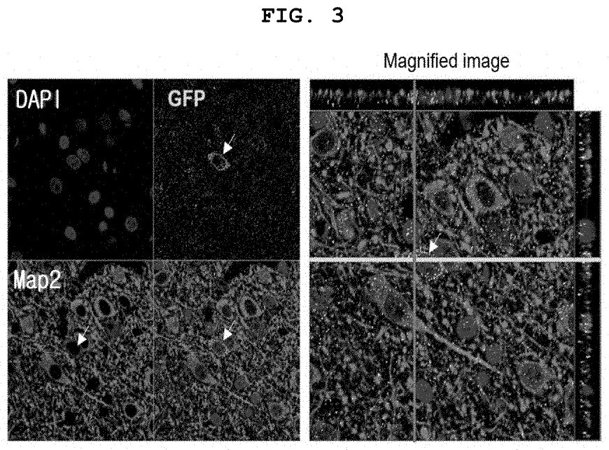

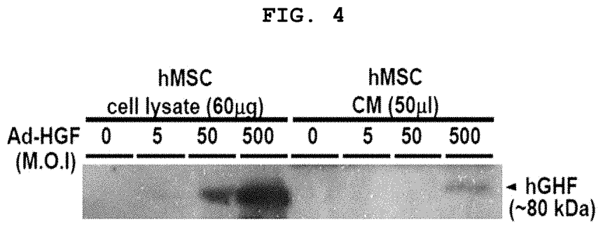

Firstly, to block non-specific interaction, the tissue section was reacted with 10% normal horse serum (NHS) at room temperature for 1 hour, and then reacted at 4° C. for 16 hours with primary antibodies of MAP2 (Microtubule-associated protein-2) antibody and GFP antibody each diluted at 1:200. After washing three times with 1×PBS/0.1% Triton X-100 for 15 minutes, the sections were allowed to react with FITC-conjugated anti-mouse IgG (Vector, 1:200) to detect the GFP primary antibody or Taxas red-conjugated anti-mouse IgG (Vector, 1:200) to detect the MAP2 primary antibody (FIG. 3). FIG. 3 is the result of immunohistochemistry using anti-neuronal marker MAP2 antibody to examine neurogenic differentiation of MSCs at two weeks after MSC/Ngn1 were infected with GFP-expressing adenovirus and transplanted into the striatum of albino rat. As shown in FIG. 3, the GFP-expressing cells and the MAP2-expressing cells were overlapped, indicating that MSC/Ngn1 were differentiated into neuronal cells.

### Example 4

### Construction and Expression of HGF Gene-Introduced Adenoviral Vector

### Example 4-1

### Construction of Adenoviral Vector Expressing HGF

The base sequence of SEQ ID NO 1 corresponding to the coding region (166-2352 bp) in the gene sequence of GenBank Accession NO NM_000601.4 was introduced into a pShuttle-CMV vector to prepare a pShuttle-CMV-HGF. This vector and pAdEasy-1 were co-transduced into E. coli (BJ 5183 strain) by electroporation, and cultured in a medium containing kanamycin (50 μg/mL) until colonies were formed. Plasmids were obtained from each colony, and candidate colonies were selected by standard restriction enzyme digestion. The base sequence was analyzed to obtain a pAd-HGF vector having HGF. The pAd-HGF was linearized by cleavage with the restriction enzyme PacI, and introduced into HEK293 cell by calcium phosphate precipitation to obtain a culture broth containing Adeno-HGF virus.

### Example 4-2

### Western Blot Analysis on HGF Expression in Adenovirus

In order to examine whether HGF was normally expressed in the adenovirus introduced with HGF gene, MSCs were infected with the adenovirus at various concentrations for 2 hours, and the produced HGF was analyzed at intracellular protein (cell lysate) and extracellular protein (conditioned-medium; CM) levels by Western blotting (FIG. 4). FIG. 4 is the result of Western blot analysis showing the expression of intracellular (cell lysate) and extracellular (conditioned-medium; CM) HGF in MSC/HGF. As shown in FIG. 4, the intracellular and extracellular HGF was produced in proportion to the concentration of HGF-expressing adenovirus infected into MSCs.

### Example 4-3

### Immunocytochemistry of Adenovirus-Mediated HGF Expression

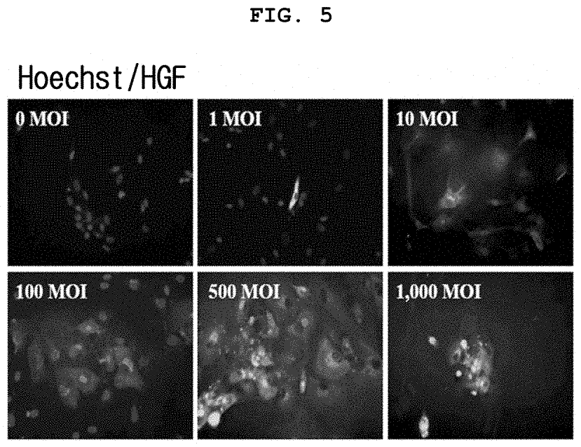

Immunocytochemistry was performed in order to examine the intracellular expression of HGF. MSCs were infected with adenovirus expressing HGF at various concentrations, fixed with 4% formalin for 10 minutes, and reacted with 10% normal goat serum (NGS) at room temperature for 1 hour to block non-specific interaction. HGF antibody diluted at 1:200 was used as a primary antibody, and reacted at 4° C. for 16 hours, followed by washing with 1×PBS/0.1% Triton X-100 for 15 minutes three times. To detect the HGF primary antibody, the cells were stained with Alexa 488-conjugated mouse Ig-G secondary antibody (Invitrogen) diluted at 1:250, and the nuclei were simultaneously stained with Hoechst (FIG. 5). FIG. 5 is a photograph showing the result of immunocytochemistry to examine the expression level of HGF in MSC/HGF. Higher MOI (multiplicity of infection) yielded higher expression of HGF (green). As shown in FIG. 5, the intracellular HGF was produced in proportion to the concentration of HGF-expressing adenovirus infected into MSCs.

### Example 5

### Introduction of HGF Gene into MSC/Ngn1 and Transplantation thereof into Stroke Animal Model

### Example 5-1

### Introduction of HGF Gene into MSC/Ngn1

MSC/Ngn1 were cultured, until the cells reached to approximately 70% confluence in a 100 mm culture plate. The transfection was carried out by adding HGF-expressing adenovirus solution obtained in Example 4 with 50 MOI for 2 hours. The MSCs were washed with PBS three times, and then MSCs were detached from the culture plate using trypsin.

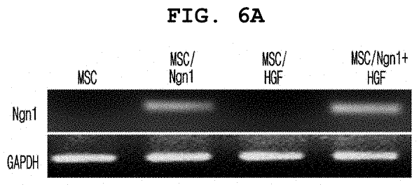

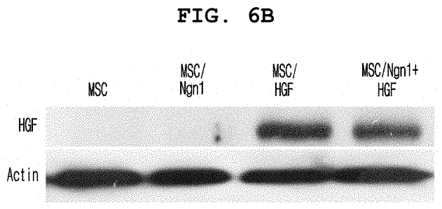

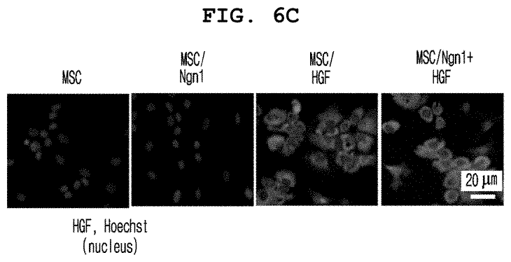

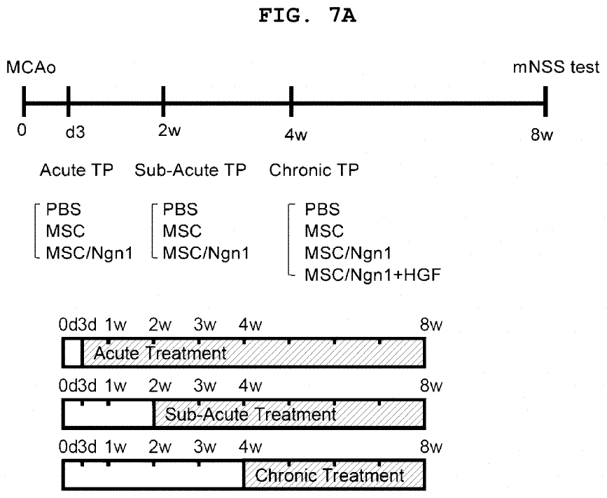

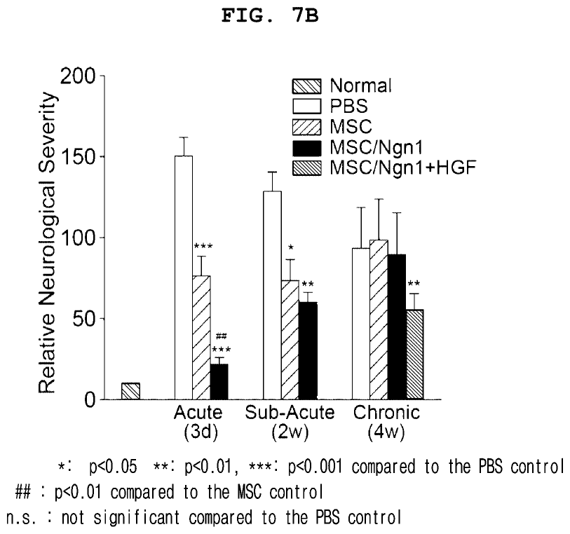

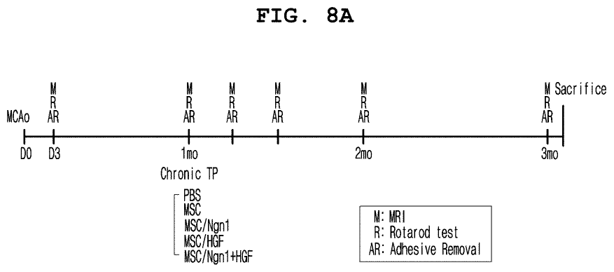

After transduction, MSC/Ngn1+HGF were confirmed by RT-PCR, western blot analysis and immunocytochemistry in order to examine the intracellular expression of Ngn1 and HGF. Two days later, expression of human neurogenin 1 was verified in MSC/Ngn1 and MSC/Ngn1+HGF by RT-PCR (FIG. 6A). GAPDH was used as internal control. Expression of HGF was verified in MSC/HGF and MSC/Ngn1+HGF by Western analysis (FIG. 6B). Actin (a ubiquitous cytoskeletal protein) was used as a loading control. Expression of HGF (red) in transduced MSC cells were verified by immunocytochemistry (FIG. 6C). Hoechst dye (blue) was used to visualize the cells. The results indicate that the MSCs were successfully engineered to express Ngn1 and HGF.

### Example 5-2

### Preparation of Stroke Animal Model

Adult male SD-rats weighing 200 g to 250 g were anesthetized with 5% isofluran gas containing 70% N2O and 30% O2. The right common carotid artery (CCA), right external carotid artery (ECA), and right internal carotid artery (ICA) were exposed through a ventral midline incision in the neck, and approximately 20 mm to 22 mm of 4-0 nylon suture was inserted from CCA to ICA to occlude the right middle cerebral artery (MCA). After 120 minutes, the nylon suture was removed. During the operation, the body temperature of the rats was maintained at 37.8° C., and all surgical instruments were sterilized before use.

### Example 5-3

### Transplantation of MSC/Ngn1+HGF into Stroke Animal Model

4 weeks after stroke induction, albino rats were placed in a stereotaxic apparatus, and 5.0×10⁵ of MSC/Ngn1+HGF were transplanted at a rate of 0.5 μL/min at positions of bregma AP=+0.5 mm, ML=3.5 mm, DV=5.0 mm and AP=−1.0 mm. ML=3.0, DV=2.5 mm using a 25-Gauge Hamilton syringe.

Five minutes after transplantation, the Hamilton syringe was removed. MSCs, MSC/Ngn1+HGF, MSC/Ngn1 and PBS were used for cell transplantation.

As shown in FIG. 7A, after ischemic stroke induced by MCAo (occlusion of middle cerebral artery), the indicated cells were transplanted at post-ischemic day 3 (d3), 2 weeks (2w) and 4 weeks (4w) representing the acute, sub-acute, and chronic stage (upper panel), respectively. Eight weeks later (8w), neurological scores were assessed by mNSS test (modified neurological severity scoring test).

### Example 5-4

### Therapeutic Effects of MSC/Ngn1+HGF on Chronic Brain Injury

FIG. 7B is a graph showing the beneficial effects of MSC/Ngn1 compared to the MSCs only in acute and subacute stages. When transplanted at 3 days (acute) and 2 weeks (subacute) after brain injury, MSC/Ngn1 lowered the neurological severity scores compared to the PBS group, or MSC group. However, such effect was not observed when MSC/Ngn1 were transplanted in the chronic phase (4 weeks after MCAo).

In contrast, MSC/Ngn1+HGF showed therapeutic effects even when transplanted 4 weeks after stroke injury (FIG. 7B). (*: p<0.05 **: p<0.01, ***: p<0.001 compared to the PBS control, ##: p<0.01 compared to the MSC control)

Note that only MSC/Ngn1+HGF can partially restore the functionality following transplantation at chronic stage (4 weeks after MCAo). Therefore, the above results suggest that MSC/Ngn1+HGF show therapeutic effects on chronic brain injury.

### Example 6

### Introduction of MSC/Ngn1+HGF and Evaluation of their Effectiveness in Stroke Animal Model

### Example 6-1

### Criteria Establishment for Evaluation of

Effectiveness of MSC in Stroke Animal Model

To evaluate the effectiveness of MSCs transplanted into animals with brain injury, an MRI and behavioral tests were performed. Stroke was induced in albino rats by middle cerebral artery occlusion (MCAo). After 4 weeks, 3.0T MRI and the behavioral tests were performed to select animals with uniform brain injury, and diverse cells were transplanted thereto.

The albino rats were anesthetized with an intraperitoneal injection of 75 mg/kg ketamine and 5 mg/kg rumpun, and an MRI scan of the rat brain was performed using a 3.0T MRI scanner equipped with a gradient system capable of 35 millitesla/m. A fast-spin echo imaging sequence was used to acquire T2-weighted anatomical images, using the following parameters: repetition time, 4,000 ms; effective echo time, 96 ms; field of view, 55×55 mm²; image matrix, 256×256; slice thickness, 1.5 mm; flip angle, 90°; number of excitations, 2; pixel size, 0.21×0.21 mm².

The relative infarct volume (RIV) was assessed using the equation RIV=[LT−(RT−RI)]×d where LT and RT represent the areas of the left and right hemispheres, respectively; RI is the infarcted area; and d is the slice thickness (1.5 mm). Relative infarct volumes were expressed as a percentage of the left hemispheric volume.

For the animal behavioral test, Adhesive Removal Test and Rotarod Test were performed. For the Adhesive Removal Tests, an adhesive tape of 10 mm×10 mm was placed on the dorsal paw of each forelimb, and the time to remove each tape from the dorsal paw was measured. For the Rotarod Test, experimental animals were tested for their ability to run on a rotating cylinder that was accelerated from 4 to 40 rpm for 5 minutes. Two weeks before stroke induction, only animals capable of removing the adhesive tape within 10 seconds and remaining on the Rota-rod cylinder for more than 300 seconds were selected and included in the experiment.

### Example 6-2

### Evaluation on Therapeutic Effectiveness of MSC/Ngn1+HGF in Stroke Animal Model

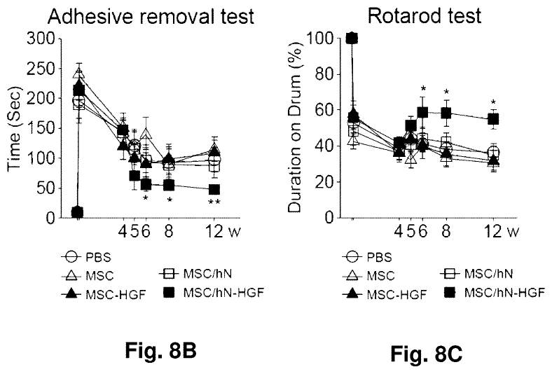

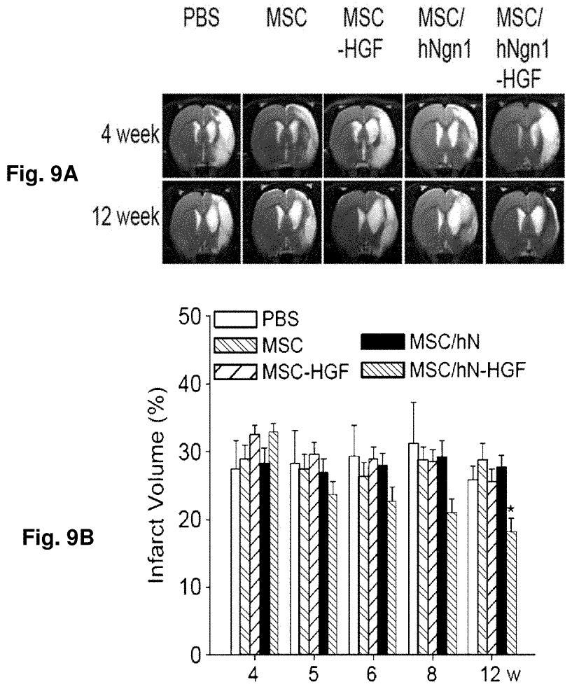

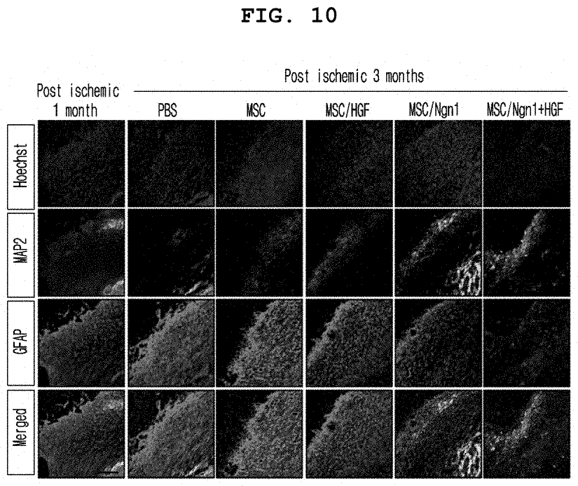

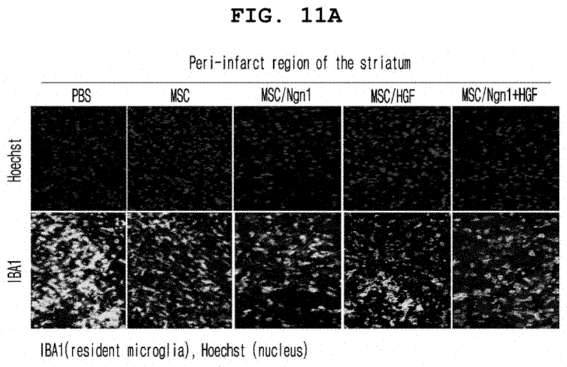

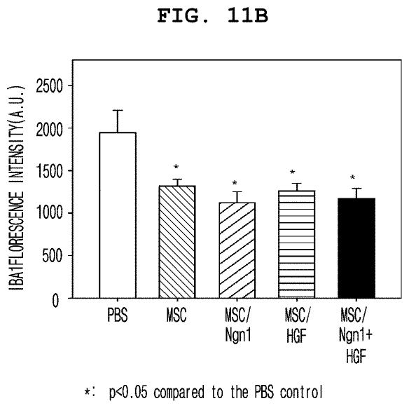

Four weeks after stroke induction, the behavioral tests and MRI were performed to select animals with uniform brain injury. The stroke animal models were transplanted with normal MSCs, MSC/HGF, MSC/Ngn1 and MSC/Ngn1+HGF. The effectiveness of the MSCs in stroke animal model was evaluated based on the behavioral tests (FIG. 8A-8B) and MRI (FIG. 9).

FIG. 8B is graphs showing the results of animal behavioral tests of Adhesive Removal Test (left panel) and Rotarod Test (right panel) to evaluate the therapeutic efficacy of MSC/Ngn1+HGF in stroke animal model.

As shown in FIG. 8A and 8B, when PBS, MSC/HGF and MSC/Ngn1 were transplanted at 4 weeks after stroke induction, no therapeutic efficacy was observed. On the contrary, transplantation of MSC/Ngn1+HGF 4 weeks after MCAo could lower the adhesive removal time while increasing the duration time on the rotating rotarod. (*: p<0.05; **: p<0.01 compared to the PBS control)

The above results suggest that transplantation of MSC/Ngn1+HGF in the chronic stroke animal model shows excellent therapeutic efficacies on motor and sensory loss caused by brain injury in stroke model.

In addition, the therapeutic efficacies of MSC/Ngn1+HGF in the stroke animal model were examined by MRl (FIG. 9). FIG. 9 is a photograph showing the results of the MRI (upper panel) and quantitative analysis of stroke lesion (lower panel) to evaluate the therapeutic efficacy of MSC/Ngn1+HGF in a chronic stroke animal model. As shown in FIG. 9, when PBS, MSC/Ngn1 or MSC/HGF were transplanted at 28 days after stroke induction, the infarct size was not reduced. On the contrary, when MSC/Ngn1+HGF were transplanted, a reduction in the infarct size was observed. (*: p<0.05 compared to the PBS control).

The above results suggest that MSC/Ngn1+HGF shows excellent therapeutic efficacies to reduce the brain infarction, compared to MSC/Ngn1.

### Example 7

### Mechanism of Therapeutic Efficacy of MSC/Ngn1+HGF in Stroke Animal Model

In order to examine the mechanism of therapeutic efficacy of MSC/Ngn1+HGF on the infarct region, tissue slices were prepared and analyzed by immunohistochemistry after completing the behavioral tests.

### Example 7-1

### Preparation of Tissue Slice

Eight weeks after transplantation (3 months after MCAo), the albino rats were anesthetized as in Example 3-4 to extract the brains. The brains were post-fixed in the fixation solution at 4° C. for 16 hours. The post-fixed brains were sectioned with a thickness of 2 mm, dehydrated in an automated tissue processor, and infiltrated with xylene and paraffin. The tissues infiltrated with paraffin were embedded with paraffin, sectioned using a rotary microtome (Leica) with a thickness of 5 μm, and mounted to silane-coated slides. As a first stage of immunohistochemistry to recover tissue antigenicity, tissues were dipped in 10 mM sodium citrate, heated using a microwave at 99° C. for 10 minutes, and cooled at room temperature for 20 minutes.

### Example 7-2

### Immunohistochemical Staining

The tissue slices prepared in Example 7-1 were dipped in 1× PBS/0.1% Triton X-100 for 30 minutes. As a first stage of immunohistochemistry, they were reacted with normal goat serum at room temperature for 1 hour to block non-specific interaction. As primary antibodies, MAP2 and GFAP antibodies (1:200 dilution) were reacted at 4° C. for 16 hours. After washing three times with 1× PBS/0.1% Triton X-100 for 15 minutes, the sections were allowed to react with Alexa 488-conjugated secondary antibody (Invitrogen, 1:250) to detect the MAP2 primary antibody and to react with Alexa 568-conjugated secondary antibody (Invitrogen, 1:250) to detect the GFAP primary antibody.

FIG. 10 is a photograph showing the result of immunohistochemistry using GFAP and MAP2 antibodies to examine glial scar (GFAP+, red) and neurons (MAP2, green), respectively. The brain from 1 month after MCAo was used as the control. When MSC, MSC/HGF, and MSC/Ngn1 were transplanted 4 weeks later MCAo, there were no changes in glial population (red) and neurons (green) at 12 weeks after stroke induction (MCAo). On the contrary, when MSC/Ngn1+HGF were transplanted, scarce distribution of glial cells was observed.

Next, the immunoreactivity of MAP2+neuronal cells (arrows,) was examined. As a result, transplantation of MSC/Ngn1+HGF elicited higher level of neuronal cells in peri-infarct region, compared to the transplantation of other cell types.

The above results suggest that combined effects of more neuronal cells together with less brain fibrosis (gliosis), MSC/Ngn1+HGF leads to higher therapeutic effects in chronic brain injury model.

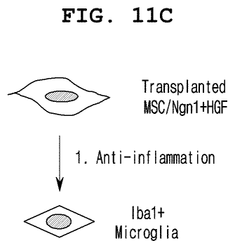

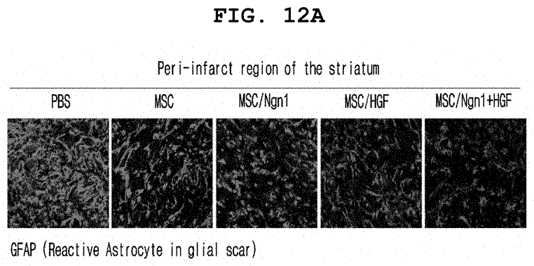

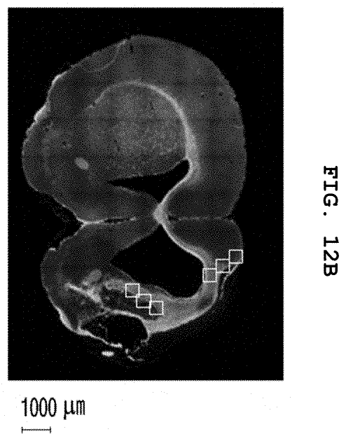

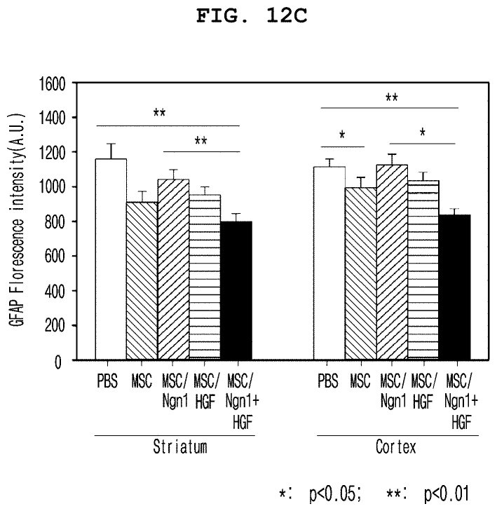

FIG. 11 is a photograph showing the brain inflammation in the ischemic brain of the animals that were sacrificed at 3 months after MCAo as shown in FIG. 8A. IBA1 (green) is a marker for resting and activated microglia (resident brain macrophages). FIG. 11B is a graph showing the IBA1-positive immunoreactivity, which was reduced following any types of transplantation (MSC, MSC/Ngn1, MSC/HGF, and MSC/Ngn1+HGF) compared to the PBS control. (*: p<0.05 compared to the PBS control). Anti-inflammatory function of MSCs is well preserved after introducing the Ngn1 gene or HGF gene as shown by suppression of the IBA1-immunoreactivity compared to the PBS control group. However, the well manifested, anti-inflammatory functions is not sufficient to improve motor deficits in the chronic phase when the brain inflammation has subsided. FIG. 11C is a schematic presentation of the anti-inflammation effects of MSC/Ngn1+HGF.

The results indicate that unlike acute phase stroke, the therapeutic effects in the chronic stroke do not solely depend on the anti-inflammatory function of MSCs.

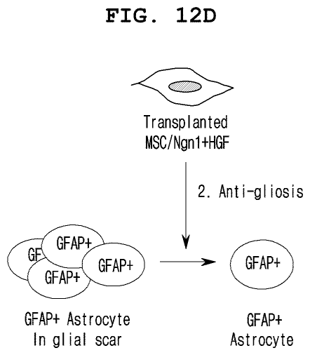

FIG. 12A is a photographs showing astrocytic glial scar (GFAP+, green) in peri-infarct region of the animals that were sacrificed at 3 months after MCAo as shown in FIG. 8A. FIG. 12B is a representative photograph showing the peri-infarct region (FIG. 12B) and the relative intensity of GFAP (red) from 3 animals per group (FIG. 12C). When MSC/Ngn1+HGF were transplanted, the distribution of glial cells was thinnest. (*: p<0.05; **: p<0.01). FIG. 12D is a schematic presentation of the anti-gliosis effects of MSC/Ngn1+HGF.

The results suggest that therapeutic effects of MSC/Ngn1+HGF is due in part to the resolution of glial scar that is known to interfere with axonal regeneration.

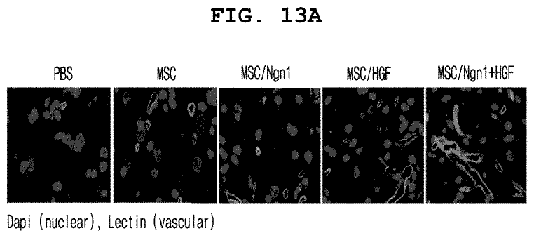

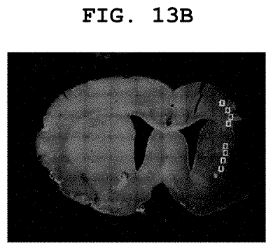

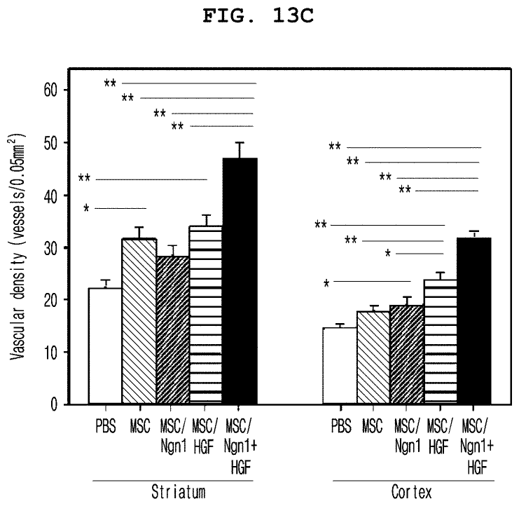

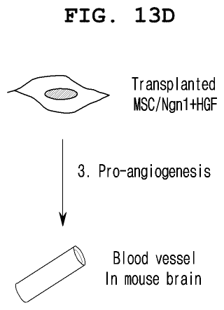

FIG. 13A is a photograph showing distribution of blood vessels in the brain of the animals that were sacrificed at 3 months after MCAo as shown in FIG. 8A. Blood vessels were visualized with Tomato-Lectin (1:500, Sigma Aldrich, red). FIG. 13B is a photograph showing the area of interest in the peri-infarct region of the striatum and cortex. Images of blood vessels labeled with Tomato Lectin was acquired from 8 boxes in the peri-infarct region of 3 animals per group.(*: p<0.05; **: p<0.01). FIG. 13C is a relative intensity of Tomato lectin labeled-blood vessels in the striatum and cortex. FIG. 13D is a schematic presentation of the pro-angiogenic effect of MSC/Ngn1+HGF.

Importantly, transplantation of MSC/Ngn1+HGF is the most effective to enhance the blood vessel density. The results suggest that therapeutic effect of MSC/Ngn1+HGF may be due in part to increased angiogenesis (blood vessel formation) in the brain, which support proliferation of endogenous neural precursor cells.

In order to assess neurogenesis following transplantation in a mouse chronic model generated by MCAo, cells were transplanted 1 month after MCAo and then Bromo-deoxyuridine (BrdU), a thymidine analog, was intraperitoneally injected (50 mg/kg/day) for 5 consecutive days from day 32 (two days after transplantation) after MCAo to trace proliferating cells in the chronic phase. On day 38 after MCAo, animals were sacrificed, and brain sections were analyzed by immunohistochemical methods.

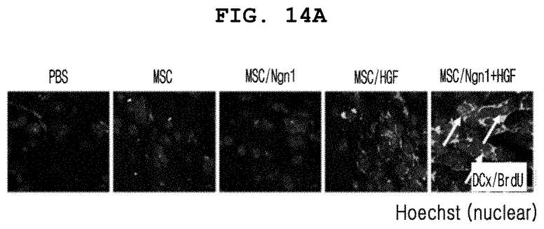

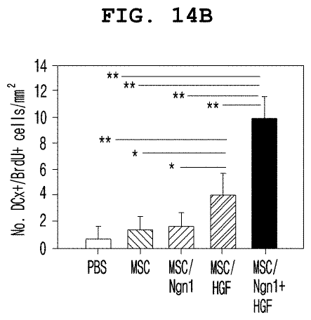

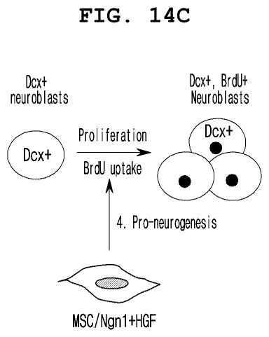

FIG. 14A and 14B shows that the number of Dcx+ (Doublecortin-positive) neuroblasts were significantly increased in the striatum of the animals transplanted with MSC/Ngn1+HGF. The Dcx+ cells were labeled by BrdU, indicating the proliferation of endogenous neuroblasts after transplantcation. In contrast, the effects of MSC and MSC/Ngn1 were minimal, while MSC/HGF were less effective to increase DCx+ cells in a chronic stroke model. (*: p<0.05; **: p<0.01) The results suggest that therapeutic effects of MSC/Ngn1+HGF is due to the increased proliferation of DCx+ endogenous neuroblasts located near the transplantation site. FIG. 14C is a schematic presentation of the pro-neurogenic effects of MSC/Ngn1+HGF.

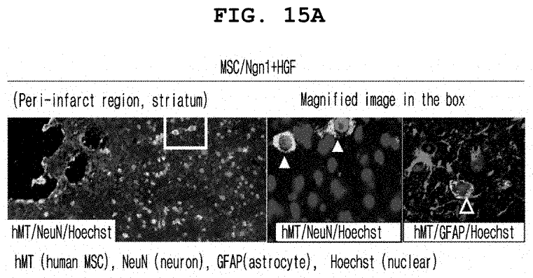

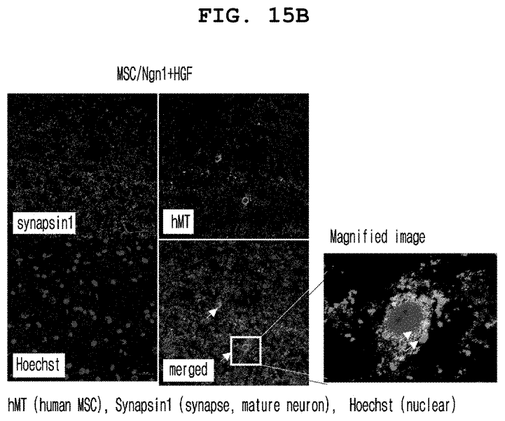

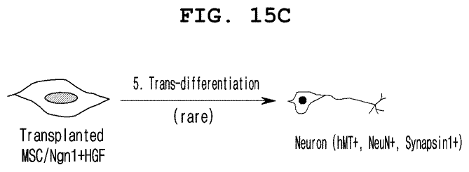

Two months after transplantation (3 months after MCAo), only MSC/Ngn1+HGF, but no other cell types were detected. FIG. 15A show that some remaining MSC/Ngn1+HGF (human mitochondrial antigen, hMT+ green) acquired neuronal phenotype (NeuN+, white arrowheads). MSC/Ngn1+HGF never became astrocytes (GFAP+, open arrowheads). FIG. 15B shows that MSC/Ngn1+HGF (green) were occasionally positive for Synasin 1 (a synaptic marker). FIG. 15C is a schematic presentation of trans-differentiation of MSC/Ngn1+HGF.

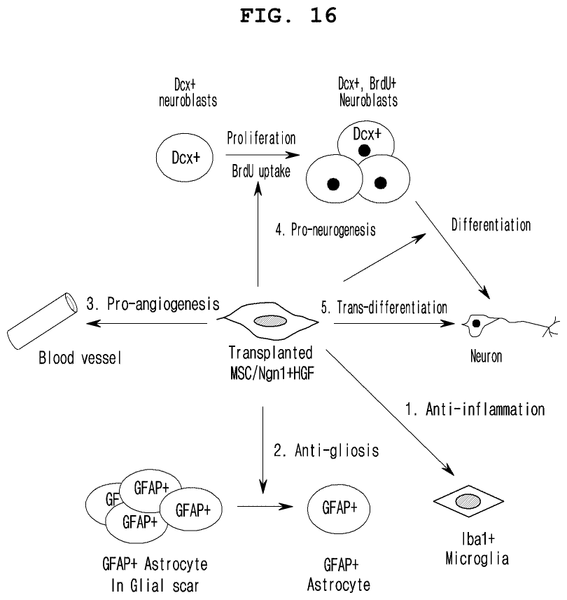

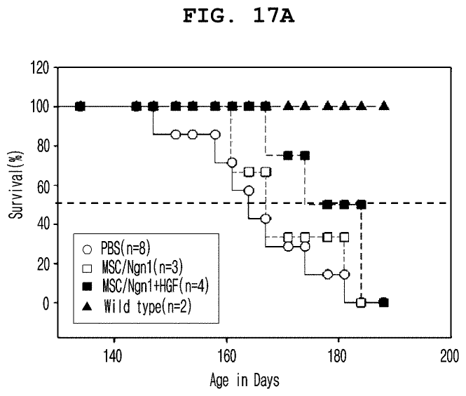

The results suggest that therapeutic effect of MSC/Ngn1+HGF may be due in part to their beneficial functions (pro-angiogenesis, pro-neurogenesis, anti-gliosis, anti-inflammation) as well as reconstitution of neural network with host neurons via trans-differentiation into functional neurons, as shown in FIG. 16.

### Example 8

### Transplantation of MSC/Ngn1+HGF into ALS Animal Model

### Example 8-1

### Preparation of ALS Animal Model

Transgenic mice harboring a high copy number of the hSOD1G93A [B6SJL-TgN (SOD1-G93A)1Gur] transgene, described by Gurney et al. (Gurney, et al., Motor neuron degeneration in mice that express a human Cu, Zn superoxide dismutase mutation. Science 264: 1772-1775; 1994) exhibit degeneration of ventral motor neurons in spinal cord and thus are commonly used as an ALS model. The transgenic hSOD1G93A males were obtained from Jackson Laboratories (Bar Harbor, Me., USA) and maintained by crossing with F1 hybrid females obtained from C57BL6 females with Swiss Jim Lambert (SJL) males. Genotypes were verified by polymerase chain reaction (PCR) using genomic DNA isolated from mouse tail extracts.

### Example 8-2

### Transplantation of HGF Gene and Neurogenin 1 Gene-Introduced MSCs into ALS Animal Model

1×10⁶ cells each of MSC/Ngn1 and MSC/Ngn1+HGF were transplanted into tail veins in a week that animals first failed the paw grip endurance (PaGE) test (13th-14th weeks). PaGE test measures the latency to fall for a mouse holding onto the inverted lid of a cage and allows early detection of disease onset. Each mouse was given three trials, and the longest latency was recorded. The cutoff time was 90 s. PBS was used as vehicle control.

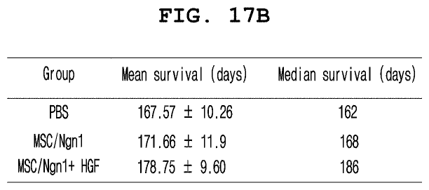

FIG. 17A is a graph showing that the survival of animals was increased by transplantation of cells. PBS was used as a negative control. FIG. 17B is a summary showing that both the means and median was increased by the transplantation of the cells. MSC/Ngn1 slightly increased the means and median by 4 and 6 days, respectively. By comparison, MSC/Ngn1+HGF increased the means and median by 11 and 26 days compared to the PBS control. Since this animal model carries a high copy number of G93A mutated SOD1, the overall survival remains unchanged. Therefore, the prolongation of median survival days by 26 days suggests that MSC/Ngn1+HGF can be an effective treatment for sporadic ALS patients who are more frequent than those with familial ALS.

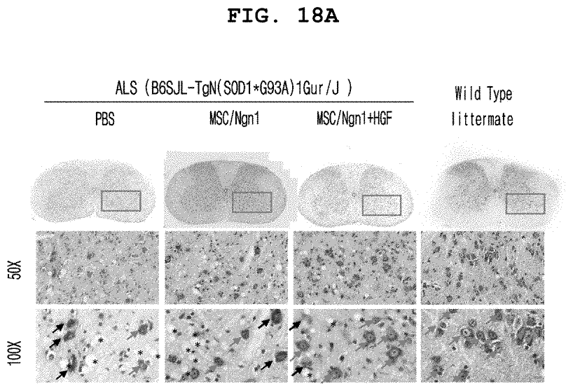

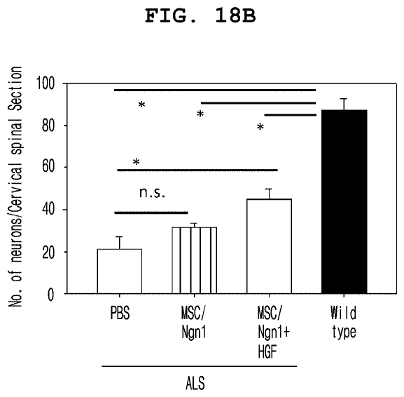

FIG. 18A is a photograph showing the cross section of spinal cord stained with cresyl violet. The images in red boxes were magnified to assess the ventral motor neurons. Healthy motor neurons were found to have euchromatic nuclei with prominent nucleoli (red arrows) whereas apoptotic cells exhibited dense, pyknotic nuclei (black arrows). Wild type littermates were used as a positive control, whereas PBS injected ALS animals were used as negative control. FIG. 18B is a summary graph showing the number of healthy ventral motor neurons. Data indicate means±SEM from two sections each per animal and three animals per group. The number of motor neurons were highest in the animals with MSC/Ngn1+HGF (*: p<0.05; n.s.: not significant).

### Example 9

Transplantation of MSC/Ngn1+HGF into AD Animal Model

### Example 9-1

### Preparation of AD Animal Model

5xFAD transgenic mice were used to assess the therapeutic effects of the transplanted cells on learning and memory. These transgenic mice carry a human APP (amyloid precursor protein) with Swedish (K670N, M671L), Florida (1716V), and London (V717I) mutations and a human PS1 (presenilin 1) with M146L and L286V mutations and recapitulate major features of Alzheimer's disease. Male hemizygous transgenic mice with 5xFAD mutations were obtained from Jackson Laboratories and maintained by crossing hemizygous transgenic mice with B6SJL F1 mice. Genotypes were verified by polymerase chain reaction (PCR) using genomic DNA isolated from mouse tail extracts.

### Example 9-2

Transplantation of MSC/Ngn1+HGF into AD Animal Model

About 24 week old 5xFAD mice were divided into three groups with 5-6 mice per group. 1.5×10⁵ cells in 1.5 μl PBS were transplanted bilaterally into the dentate gyrus with stereotaxic coordinates of AP: −1.06 mm; ML: ±1.0 mm; DV: −2.5 mm for 15 min. The third group that was injected with PBS and the non-transgenic wild type littermates were used as controls.

Six weeks after the transplantation, the therapeutic potentials to treat Alzheimer's disease were assessed by Morris water maze test which has been widely used to test learning and memory functions of rodents. The water maze apparatus consisted of a circular pool with 140 cm in diameter and 45 cm in height. The apparatus was filled with 21-23° C. opaque water by adding dry milk powder that helped the animal to hide the submerged platform. The top surface of the hidden platform was 1.5 cm below the water surface. Four distinct visual cues were given in 4 locations (N, S, E, W) on the sidewall of the apparatus. Animals were placed in water facing the visual cues on the sidewall at each starting point of three quadrants. Three starting points were changed daily. Animals were required to find a submerged platform in the pool by using those spatial cues. Spatial training consisted of 5 consecutive days and 3 trials with different starting points per session per day. Throughout the session the platform was left in the same position and the latency to escape on to the hidden platform was recorded in each training session. The results are the mean swimming time traveled per trial toward the platform. The mean values for 5 days from 3 trials of 5-6 animals per group are shown.

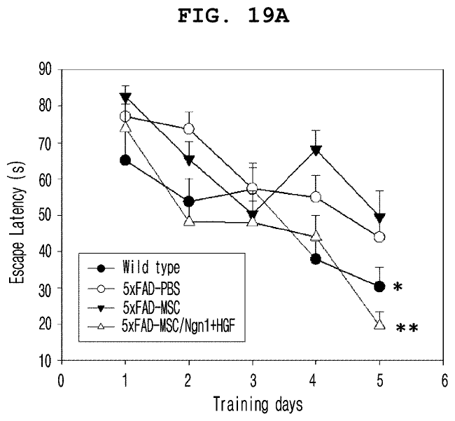

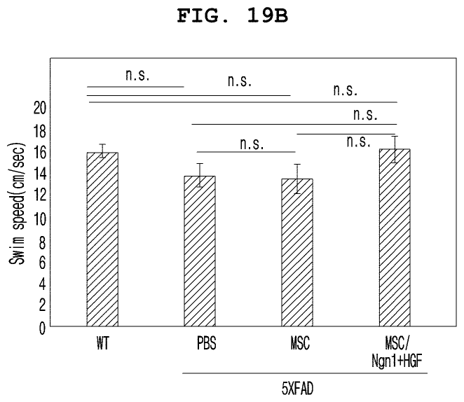

As shown in FIG. 19A, wild type mice learned to find a platform during test period and immediately found a hidden platform at 5th day. In contrast, 5xFAD mice with PBS as vehicle control showed poor performance in learning to escape to the hidden platform (compare 30.3±5.4 sec for the wild type and 43.9±5.4 sec 5X FAD-PBS control at day 5). However, the 5xFAD mice with MSC/Ngn1+HGF acquired spatial memory, which was comparable to the level of the wild type littermates at day 5 (19.5 ±4.0 sec). In contrast, the 5xFAD mice with naive MSC showed poor performance (49.6 ±7.1 sec), which was similar to the level found in the 5xFAD-PBS. The results indicated that there was a significant enhancement in learning following transplantation of MSC/Ngn1/HGF and this enhancement was not achievable with naive MSC. (*: p<0.05; **: p<0.01 compared to the PBS control.)

As shown in FIG. 19B, swimming speed, not a cognitive factor, was not significantly different among the groups compared to the wild type. The results indicate that the different escape latency shown in FIG. 19A is due to different cognitive ability. The results suggest that MSC/Ngn1+HGF can be a therapeutic strategy to improve cognitive functions in AD patients. (n.s.: not significant).

### Example 9-4

### Assessment for Mechanism Underlying Therapeutic Effect of MSC/Ngn1+HGF in AD Animal Model

To further assess the mechanism underlying therapeutic effects of MSC/Ngn1+HGF in an AD mouse model, the brains were isolated after completing Morris water maze test and subject to immunohistochemistry.

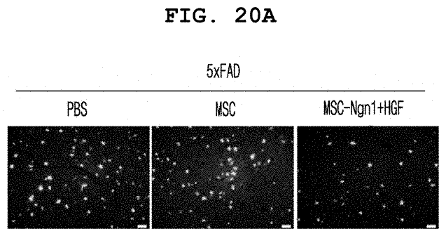

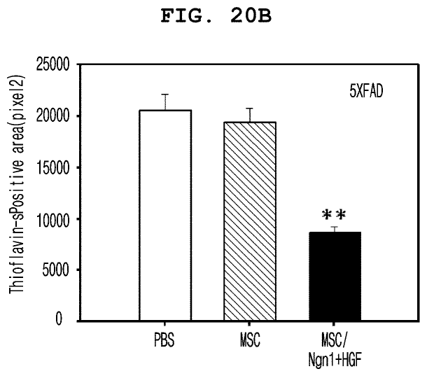

As shown in FIG. 20A, the thioflavin-positive β-amyloid plaques were less produced in the brain after transplantation of MSC/Ngn1+HGF in 7 months-old 5xFAD mice. Transplantation of MSC/Ngn1+HGF could reduce the thioflavin-S positive plaques while that of MSCs could not. In the wild type control, thioflavin-S positive plaques were not found.

In addition, as shown in FIG. 20B, the thioflavin-positive pixels obtained from 12 brain sections from three animals per group were captured and quantified using Image J software. The results reveal that the superior functions of the animals transplanted with MSC/Ngn1+HGF in the Morris water maze test is partly ascribed to the delay of the disease progression in 5xFAD mice. (**: p<0.01 compared to the PBS control.)

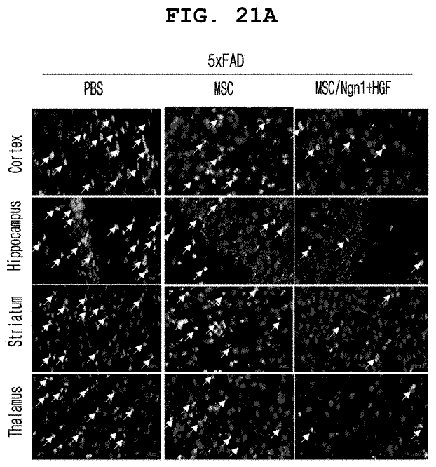

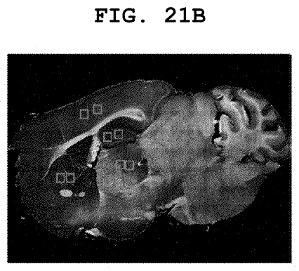

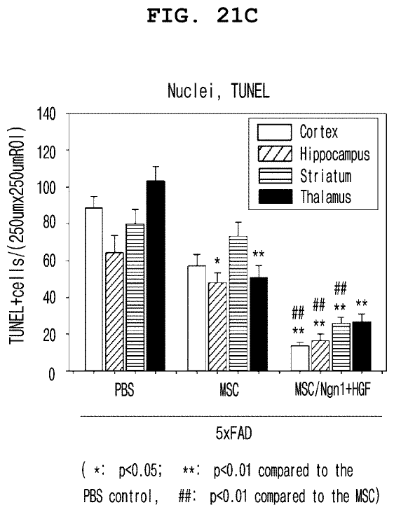

FIG. 21A is a photograph showing apoptotic cells in the representative regions of interest (red boxes) in cortex, hippocampus, striatum, and thalamus in a parasagittal section of the brain as shown in FIG. 21B. No apoptotic cells were found in the wild type littermates, indicating that the apoptotic cell death is indicative of disease progress in 5xFAD mouse model. Images from regions of interest were analyzed using ZEN software (Blue Edition, Zeiss). FIG. 21C is a graph showing the quantitative data of apoptotic cells from four independent regions of interest in two sections per mice, three mice per group. The results indicate that apoptosis is the lowest in animals grafted by MSC/Ngn1+HGF. (*: p<0.05; **: p<0.01 compared to the PBS control, #: p<0.05; ##: p<0.01 compare to the MSC control)

The results indicate that the cognitive activity in 5xFAD mice (FIG. 19A) inversely correlates well with the level of amyloid plaques (Thioflavin+) as shown in FIG. 20 and apoptotic cell death (TUNEL+) as shown in FIG. 21.

Taken together, the results suggest MSC/Ngn1+HGF may improve the cognitive activity by preventing accumulation of amyloid plaques and thereby prohibiting the apoptotic cell death.

1. A modified mesenchymal stem cell, comprising a mesenchymal stem cell having introduced therein:
a gene encoding a hepatocyte growth factor (HGF); and
a gene encoding neurogenin 1.

2. The modified mesenchymal stem cell of claim 1, wherein the mesenchymal stem cell is derived from one or more tissues selected from the group consisting of bone marrow, blood, umbilical cord blood, umbilical cord, adipose tissue, liver, skin, gastrointestinal tract, muscle, placenta and uterus, adult bone marrow, adult blood, adult adipose tissue, liver, skin, gastrointestinal tract, muscle, placenta and uterus.

3. The modified mesenchymal stem cell of claim 1, wherein the mesenchymal stem cell is derived from bone marrow.

4. The modified mesenchymal stem cell of claim 1, wherein:
the gene encoding HGF comprises a nucleic acid sequence of SEQ ID NO 1;
the gene encoding neurogenin 1 comprises a nucleic acid sequence of SEQ ID NO 2; or the gene encoding HGF comprises a nucleic acid sequence of SEQ ID NO 1 and the gene encoding neurogenin 1 comprises a nucleic acid sequence of SEQ ID NO 2.

5. The modified mesenchymal stem cell of claim 1, wherein:
the gene encoding HGF is on an extrachromosomal element; or
the gene encoding HGF is on an extrachromosomal element and the extrachromosomal element is an adenoviral vector.

6. The modified mesenchymal stem cell of claim 1, wherein the mesenchymal stem cell is a human, adult mesenchymal stem cell or a stem cell derived from a human adult.

7. A method of preparing the modified mesenchymal stem cell of claim 1, the method comprising:
introducing a gene encoding hepatocyte growth factor (HGF) and introducing a gene encoding neurogenin 1 into a mesenchymal stem cell;
selecting the modified mesenchymal stem cell that is introduced with the gene encoding HGF and the gene encoding neurogenin 1; and
culturing the selected modified mesenchymal stem cell.

8. The method of claim 7, wherein:
the gene encoding HGF comprises a nucleic acid sequence of SEQ ID NO 1;
the gene encoding neurogenin 1 comprises a nucleic acid sequence of SEQ ID NO 2; or the gene encoding HGF comprises a nucleic acid sequence of SEQ ID NO 1 and the gene encoding neurogenin 1 comprises a nucleic acid sequence of SEQ ID NO 2.

9. The method of claim 7, wherein introducing the gene encoding HGF and introducing the gene encoding neurogenin 1 are performed sequentially, or in reverse order.

10. The method of claim 7, wherein the mesenchymal stem cell is derived from one or more tissues selected from the group consisting of bone marrow, blood, umbilical cord blood, umbilical cord, adipose tissue, liver, skin, gastrointestinal tract, placenta, and uterus.

11. The method of claim 7, wherein the mesenchymal stem cell is a human, adult mesenchymal stem cell or a stem cell derived from a human adult.

12. The method of claim 7, wherein the gene encoding HGF is introduced into the mesenchymal stem cell by an adenoviral vector.

13. A method, comprising administering the modified mesenchymal stem cell of claim 1 to a subject.

14. The method of claim 13, wherein administering comprises transplanting the modified mesenchymal stem cell into the brain of the subject.

15. The method of claim 13, wherein the subject is a mammal.

16. The method of claim 13, wherein the subject is diagnosed with a neurological disease.

17. The method of claim 16, wherein the neurological disease is selected from the group consisting of Alzheimer disease (AD) and amyotrophic lateral sclerosis (ALS).

18. A method of treating a neurological disease, the method comprising administering the modified mesenchymal stem cells of claim 1 to a subject having the neurological disease.

19. The method of claim 18, wherein the neurological disease is selected from the group consisting of Parkinson's disease, AD (Alzheimer disease), Huntington's chorea, ALS (amyotrophic lateral sclerosis), epilepsy, schizophrenia, acute stroke, chronic stroke, spinal cord injury and chronic brain injury after stroke.

20. A method of preparing a culture of modified mesenchymal stem cells, comprising:
introducing a gene encoding hepatocyte growth factor (HGF) and introducing a gene encoding neurogenin 1 into a cultured mesenchymal stem cell to produce a modified mesenchymal stem cell;
selecting the modified mesenchymal stem cell that is introduced with the gene encoding HGF and the gene encoding neurogenin 1; and
culturing the selected modified mesenchymal stem cell.
